(19) United States
(12) Patent Application Publication
(10) Pub. No.: US 2026/0069833 A1
(43) Pub. Date: Mar. 12, 2026

(54) DEFLECTABLE CATHETER WITH ASYMMETRIC ARTICULATION JOINT

(71) Applicant: Boston Scientific Scimed, Inc., Maple Grove, MN (US)

(72) Inventors: Joseph Geissler, St. Louis Park, MN (US); Brent Dolan, Blaine, MN (US); Raymond David Gessler, III, Roberts, WI (US)

(21) Appl. No.: 19/327,409

(22) Filed: Sep. 12, 2025

Related U.S. Application Data
(60) Provisional application No. 63/694,109, filed on Sep. 12, 2024.

(51) Int. Cl. A61M 25/01; A61B 18/00; A61M 25/00

(52) U.S. Cl. CPC A61M 25/0136; A61B 18/00; A61M 25/0052; A61M 25/0138; A61B 2018/00184; A61B 2018/0091

(57) ABSTRACT

A medical device includes a handle and a tubular shaft extending from the handle, and a distal portion having a deflection region. The shaft includes a jacket and an articulation member within the jacket. The articulation member has a proximal and distal articulation regions and comprises longitudinally-arranged tubular segments and connecting segments, wherein adjacent tubular segments are joined by respective connecting segments, and wherein the connecting segments are disposed in a first plane, and wherein diametrically opposed slit pairs are disposed longitudinally along the articulation member, each slit pair separating adjacent tubular segments and being centered on a second plane that is orthogonal to the first plane. The proximal articulation region is configured to articulate in a first direction and to resist articulation in a second, opposite direction. The distal articulation region is configured to articulate in both the first and second directions.

### CROSS REFERENCE TO RELATED APPLICATIONS

This application claims priority to U.S. Provisional Patent Application No. 63/694,109 entitled “DEFLECTABLE CATHETER WITH ASYMMETRIC ARTICULATION JOINT,” filed September 12, 2024, which is hereby incorporated by reference in its entirety.

### TECHNICAL FIELD

The present invention relates to medical devices and methods for catheters for medical procedures. More specifically, the invention relates to devices and methods that include directional enhancement for catheters such as steerable catheters.

### BACKGROUND

Various medical procedures involve catheters inserted into a patient's vasculature. In certain procedures, the catheter may be navigated through the vasculature to a target location in the body. The distal end of the catheters may be inserted into the patient's heart chambers in, for example, interventional electrophysiology procedures. The distal end of the catheter may include one or more electrodes that are used to delivery therapy (e.g., ablation) or map the surface of the heart tissue (e.g., identify the locations of heart tissue that are a source of the arrhythmias). Being able to steer the catheter using the distal end portion of the catheter, for example, the through various patient anatomy, may facilitate catheter performance.

### SUMMARY

In Example 1, a medical device comprising a handle; and a tubular shaft having a proximal portion extending from the handle, and a distal portion having a distal end and a deflection region, the shaft defining a longitudinal axis and including: an outer tubular jacket; and an articulation member disposed within the jacket in the deflection region, the articulation member having a proximal articulation region and a distal articulation region and comprising a plurality of longitudinally-arranged tubular segments, a plurality of first connecting segments, and a plurality of second connecting segments, wherein adjacent tubular segments are joined by respective ones of the first and second connecting segments, and wherein all of the first and second connecting segments are disposed in a first plane extending through the longitudinal axis, and wherein a plurality of diametrically opposed slit pairs are disposed longitudinally along the articulation member, each slit pair separating adjacent tubular segments between respective ones of the first and second connecting segments and including a first slit and a second slit, wherein the slit pairs are centered on a second plane that is orthogonal to the first plane; and wherein the proximal articulation region is configured to articulate in a first direction within the second plane, and to resist articulation in a second direction within the second plane, the second direction being opposite the first direction, and wherein the distal articulation region is configured to articulate in both the first direction and the second direction.

In Example 2, the medical device of Example 1, further comprising first and second steering wire lumens extending through the tubular shaft to a location distally beyond the articulation member and within the second plane; and first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

In Example 3, the medical device of Example 2 wherein the articulation member includes a first reinforcing member and a second reinforcing member extending through the plurality of first and second connecting segments, respectively, wherein the first and second reinforcing members are embedded, respectively, within the first and second connecting segments and within the tubular segments.

In Example 4, the medical device of Example 3, wherein the deflection region is configured to assume a first curved shape when the first deflection force is applied to the distal portion of the shaft; and the deflection region is configured to assume a second curved shape when the second deflection force is applied to the distal portion of the shaft, wherein the second curved shape is different than the first curved shape.

In Example 5, the medical device of Example 4, wherein the first curved shape has a first curve diameter, and the second curved shape has a second curve diameter, wherein a first radius of curvature is greater than a second radius of curvature.

In Example 6, the medical device of Example 3, wherein the articulation member is configured such that the deflection region begins to articulate in the first direction proximate a proximal end of the deflection region when the first deflection force is applied to the distal portion of the shaft; and the deflection region begins to articulate in the second direction at a transition between the proximal articulation region and the distal articulation region when the second deflection force is applied to the distal portion of the shaft.

In Example 7, the medical device of Example 3, wherein the articulation member is configured such that the deflection region begins to articulate in the first direction at a first location along the shaft when the first deflection force is applied to the distal portion of the shaft; and the deflection region begins to articulate in the second direction at a second location along the shaft when the second deflection force is applied to the distal portion of the shaft.

In Example 8, the medical device of Example 7, wherein the first location is proximal to the second location.

In Example 9, the medical device of any of Examples 1-8, wherein each of the slit pairs includes a first slit having a first slit width located at a first circumferential side of the articulation member, and a second slit having a second slit width located on a second circumferential side of the articulation member.

In Example 10, the medical device of Example 9, wherein the slit pairs in the proximal articulation region are configured to permit articulation of the proximal articulation region in the first direction within the second plane, and to resist articulation of the proximal articulation region in the second direction within the second plane.

In Example 11, the medical device of Example 9, wherein when the deflection region is in an undeflected state, the second slit width is substantially zero such that adjacent tubular segments contact each other along the first circumferential side, and the first slit width larger than the second slit width.

In Example 12, the medical device of Example 10, wherein each adjacent tubular segment in the proximal deflection region includes a limiting feature that is configured to resist articulation of the proximal articulation region in the second direction.

In Example 13, the medical device of Example 11, wherein the limiting feature includes a pair of opposed extensions positioned respective adjacent tubular segments, wherein each of the extensions is configured to contact the other extension to resist articulation of the proximal articulation region in the second direction, and to move away from the other extension to allow deflection of the proximal articulation region in the first direction.

In Example 14, the medical device of Example 9, wherein the articulation member includes a coil member extending through the tubular segments of the proximal articulation region within the second plane, the coil member configured to allow articulation of the proximal articulation region in the first direction, and to resist articulation of the proximal articulation region in the second direction.

In Example 15, the medical device of Example 13, wherein the coil member assumes a close pitch configuration when the articulation member is in an undeflected state and is configured to elongate to permit articulation of the proximal articulation region in the first direction, and to resist articulation of the proximal articulation region in the second direction when the coil member is in its close pitch configuration.

In Example 16, a medical device comprising: a handle; and a tubular shaft having a proximal portion extending from the handle, and a distal portion having a distal end and a deflection region, the shaft defining a longitudinal axis and including an outer tubular jacket; and an articulation member disposed within the jacket in the deflection region, the articulation member having a proximal articulation region and a distal articulation region and comprising a plurality of longitudinally-arranged tubular segments, a plurality of first connecting segments, and a plurality of second connecting segments, wherein adjacent tubular segments are joined by respective ones of the first and second connecting segments, and wherein all of the first and second connecting segments are disposed in a first plane extending through the longitudinal axis, and wherein a plurality of diametrically opposed slit pairs are disposed longitudinally along the articulation member, each slit pair separating adjacent tubular segments between respective ones of the first and second connecting segments and including a first slit and a second slit, wherein the slit pairs are centered on a second plane that is orthogonal to the first plane; and wherein the proximal articulation region is configured to articulate in a first direction within the second plane, and to resist articulation in a second direction within the second plane, the second direction being opposite the first direction, and wherein the distal articulation region is configured to articulate in both the first direction and the second direction.

In Example 17, the medical device of Example 16, further comprising: first and second steering wire lumens extending through the tubular shaft to a location distally beyond the articulation member and within the second plane; and first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

In Example 18, the medical device of Example 17, wherein the articulation member includes a first reinforcing member and a second reinforcing member extending through the plurality of first and second connecting segments, respectively, wherein the first and second reinforcing members are embedded, respectively, within the first and second connecting segments and within the tubular segments.

In Example 19, the medical device of Example 18, wherein the deflection region is configured to assume a first curved shape when the first deflection force is applied to the distal portion of the shaft; the deflection region is configured to assume a second curved shape when the second deflection force is applied to the distal portion of the shaft, wherein the second curved shape is different than the first curved shape; and the first curved shape has a first curve diameter, and the second curved shape has a second curve diameter, wherein a first radius of curvature is greater than a second radius of curvature.

In Example 20, the medical device of Example 18, wherein the articulation member is configured such that the deflection region begins to articulate in the first direction proximate a proximal end of the deflection region when the first deflection force is applied to the distal portion of the shaft; and the deflection region begins to articulate in the second direction at a transition between the proximal articulation region and the distal articulation region when the second deflection force is applied to the distal portion of the shaft.

In Example 21, the medical device of Example 18, wherein the articulation member is configured such that the deflection region begins to articulate in the first direction at a first location along the shaft when the first deflection force is applied to the distal portion of the shaft; and the deflection region begins to articulate in the second direction at a second location along the shaft when the second deflection force is applied to the distal portion of the shaft, wherein the first location is proximal to the second location.

In Example 22, the medical device of Example 16, wherein each of the slit pairs includes a first slit having a first slit width located at a first circumferential side of the articulation member, and a second slit having a second slit width located on a second circumferential side of the articulation member.

In Example 23, the medical device of Example 23, wherein the slit pairs in the proximal articulation region are configured to permit articulation of the proximal articulation region in the first direction within the second plane, and to resist articulation of the proximal articulation region in the second direction within the second plane.

In Example 24, the medical device of Example 23, wherein: when the deflection region is in an undeflected state, the second slit width is substantially zero such that adjacent tubular segments contact each other along the first circumferential side, and the first slit width larger than the second slit width; each adjacent tubular segment in the proximal deflection region includes a limiting feature that is configured to resist articulation of the proximal articulation region in the second direction; and the limiting feature includes a pair of opposed extensions positioned respective adjacent tubular segments, wherein each of the extensions is configured to contact the other extension to resist articulation of the proximal articulation region in the second direction, and to move away from the other extension to allow deflection of the proximal articulation region in the first direction.

In Example 25, the medical device of Example 23, wherein the articulation member includes a coil member extending through the tubular segments of the proximal articulation region within the second plane, the coil member configured to allow articulation of the proximal articulation region in the first direction, and to resist articulation of the proximal articulation region in the second direction.

In Example 26, a medical device comprising: a handle; and a tubular shaft having a proximal portion extending from the handle, and a distal portion having a distal end and a deflection region, the shaft defining a longitudinal axis and including an articulation member disposed in the deflection region, the articulation member having a proximal articulation region and a distal articulation region and comprising a plurality of longitudinally-arranged tubular segments and a plurality connecting segments, wherein adjacent tubular segments are joined by respective ones of the connecting segments, and wherein all of the connecting segments are disposed in a first plane extending through the longitudinal axis, and wherein a plurality of diametrically opposed slit pairs are disposed longitudinally along the articulation member, each slit pair separating adjacent tubular segments between respective ones of the connecting segments, wherein the slit pairs are centered on a second plane that is orthogonal to the first plane, wherein: the proximal articulation region is configured to articulate in a first direction within the second plane, and to resist articulation in a second direction within the second plane, the second direction being opposite the first direction; and the distal articulation region is configured to articulate in both the first direction and the second direction; and first and second steering wire lumens extending through the tubular shaft to a location distally beyond the articulation member and within the second plane; and first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

In Example 27, the medical device of Example 26, wherein the deflection region is configured to assume a first curved shape when the first deflection force is applied to the distal portion of the shaft; and the deflection region is configured to assume a second curved shape when the second deflection force is applied to the distal portion of the shaft, wherein the second curved shape is different than the first curved shape.

In Example 28, the medical device of Example 26, wherein each of the slit pairs includes a first slit having a first slit width located at a first circumferential side of the articulation member, and a second slit having a second slit width located on a second circumferential side of the articulation member.

In Example 29, the medical device of Example 28, wherein the slit pairs in the proximal articulation region are configured to permit articulation of the proximal articulation region in the first direction within the second plane, and to resist articulation of the proximal articulation region in the second direction within the second plane.

In Example 30, the medical device of Example 28, wherein the articulation member includes a coil member extending through the tubular segments of the proximal articulation region within the second plane, the coil member configured to allow articulation of the proximal articulation region in the first direction, and to resist articulation of the proximal articulation region in the second direction.

In Example 31, a medical device comprising: an outer tubular jacket; and an articulation member disposed within the jacket, the articulation member having a proximal articulation region and a distal articulation region and comprising a plurality of longitudinally-arranged tubular segments, a plurality of first connecting segments, and a plurality of second connecting segments, wherein adjacent tubular segments are joined by respective ones of the first and second connecting segments, and wherein all of the first and second connecting segments are disposed in a first plane extending through the longitudinal axis, and wherein a plurality of diametrically opposed slit pairs are disposed longitudinally along the articulation member, each slit pair separating adjacent tubular segments between respective ones of the first and second connecting segments and including a first slit and a second slit, wherein the slit pairs are centered on a second plane that is orthogonal to the first plane; and wherein the proximal articulation region is configured to articulate in a first direction within the second plane, and to resist articulation in a second direction within the second plane, the second direction being opposite the first direction, wherein the distal articulation region is configured to articulate in both the first direction and the second direction, and wherein each of the slit pairs includes a first slit having a first slit width located at a first circumferential side of the articulation member, and a second slit having a second slit width located on a second circumferential side of the articulation member.

In Example 32, the medical device of Example 31, wherein the slit pairs in the proximal articulation region are configured to permit articulation of the proximal articulation region in the first direction within the second plane, and to resist articulation of the proximal articulation region in the second direction within the second plane.

In Example 33, the medical device of Example 32, wherein when the articulation member is in an undeflected state, the second slit width is substantially zero such that adjacent tubular segments contact each other along the first circumferential side, and the first slit width larger than the second slit width.

In Example 34, the medical device of Example 31, wherein the articulation member includes a coil member extending through the tubular segments of the proximal articulation region within the second plane, the coil member configured to allow articulation of the proximal articulation region in the first direction, and to resist articulation of the proximal articulation region in the second direction.

In Example 35, the medical device of Example 30, further comprising: first and second steering wire lumens extending through the tubular shaft to a location distally beyond the articulation member and within the second plane; and first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is an illustration of an example electrophysiology catheter, consistent with various aspects of the present disclosure.

FIG. 2 is an illustration of the electrophysiology catheter, shown in FIG. 1, as deflected in a first direction, consistent with various aspects of the present disclosure.

FIG. 3 is an illustration of the electrophysiology catheter, shown in FIGS. 1-2, as deflected in a second direction, consistent with various aspects of the present disclosure.

FIG. 4 is a side-view illustration of a portion of a shaft of the electrophysiology catheter including a deflection region, consistent with various aspects of the present disclosure.

FIG. 5A is a top-view illustration of an articulation member of the electrophysiology catheter, consistent with various aspects of the present disclosure.

FIG. 5B is a cross-sectional illustration of a tubular segment of the electrophysiology catheter, consistent with various aspects of the present disclosure.

FIG. 5C is a side-view illustration of an articulation member of the electrophysiology catheter, consistent with various aspects of the present disclosure.

FIG. 5D is a detailed illustration of a portion of a side-view illustration of an articulation member of the electrophysiology catheter, consistent with various aspects of the present disclosure.

FIG. 6 is another illustration of an example electrophysiology catheter, consistent with various aspects of the present disclosure.

FIG. 7A is an embodiment of a side-view illustration of a portion of an articulation member of the electrophysiology catheter, consistent with various aspects of the present disclosure.

FIG. 7B is a side-view illustration of a portion of an alternative embodiment of an articulation member of the electrophysiology catheter, consistent with various aspects of the present disclosure.

FIG. 8A is a side-view illustration of a portion of an alternative embodiment of an articulation member of the electrophysiology catheter, consistent with various aspects of the present disclosure.

FIG. 8B is a detailed illustration of a portion of a side-view illustration of an alternative embodiment of an articulation member of the electrophysiology catheter, consistent with various aspects of the present disclosure.

FIGS. 9A and 9B illustrate an alternative embodiment of a tubular shaft of an electrophysiology catheter, consistent with various aspects of the present disclosure.

FIG. 10A is a side-view illustration of a portion of an alternative embodiment of an articulation member of the electrophysiology catheter, consistent with various aspects of the present disclosure.

FIG. 10B is a perspective view illustration of an alternative embodiment of an articulation member of the electrophysiology catheter, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Various aspects of the present disclosure are directed toward directional enhancement of catheters and steerable catheters. When arranged within a patient, catheters and steerable catheters may be steered or curved in a number of directions. The present disclosure includes aspects that facilitate and enhance directionality of the catheters and steerable catheters. As described in further detail below, at least portion of the catheters and steerable catheters may include directional enhancement features that bias the catheters and steerable catheters in a desired direction when curved. Furthermore, the directional enhancement features can vary to provide different curvatures in different directions to accommodate various patient anatomy.

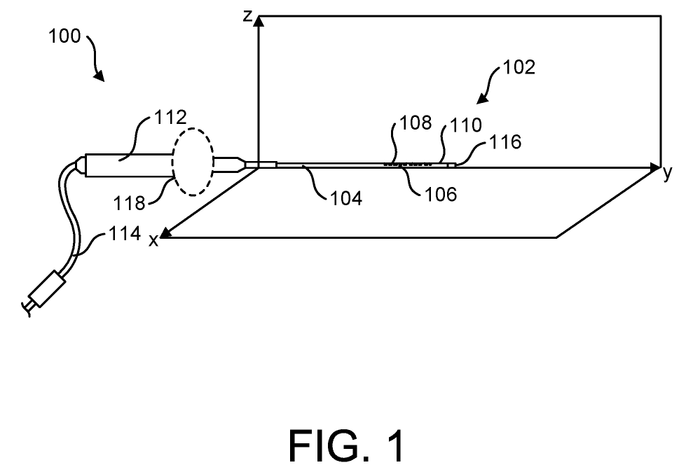

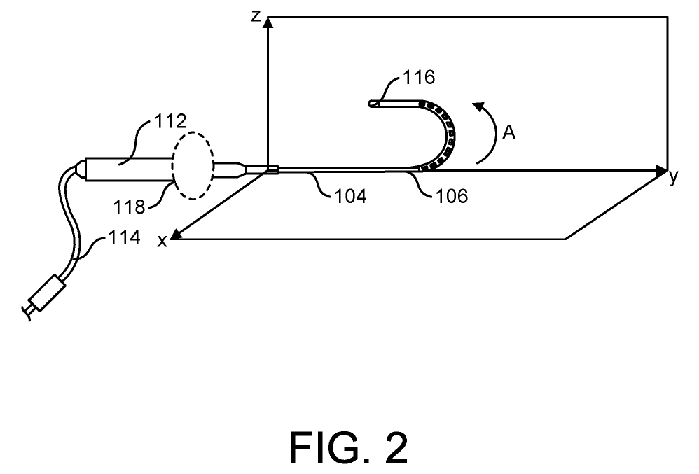

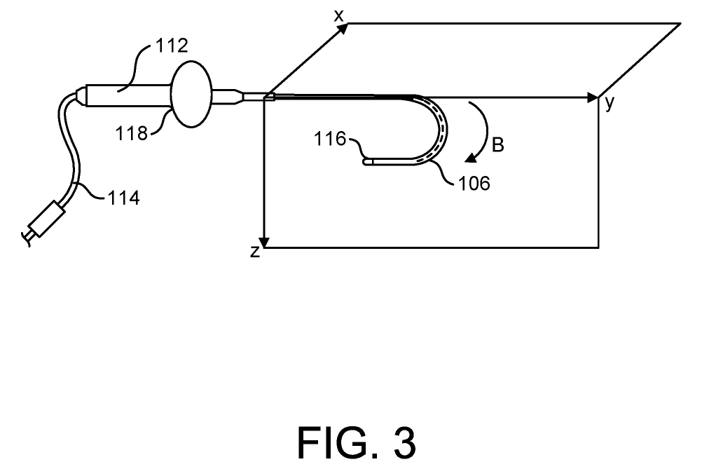

FIGS. 1-3 are illustrations of an example electrophysiology catheter 100, consistent with various aspects of the present disclosure. As shown in FIG. 1, the electrophysiology catheter 100 may be a steerable electrophysiology catheter 100. In certain instances, the electrophysiology catheter 100 is steerable in one direction (e.g., direction A as shown in FIG. 2) or in multiple directions (e.g., directions A and B as shown in FIGS. 2-3). The electrophysiology catheter 100 generally includes a tubular shaft 102 having a proximal portion 104 and a distal portion 106 that is sized and configured for placement and manipulation within in a target area of a heart of patient. The distal portion 106 may be steerable. As shown, the distal portion 106 further includes a deflection region 108 and a distal end 110.

The tubular shaft 102, in an undeflected state, defines a longitudinal axis, which may generally correspond to the geometrical centerline of the tubular shaft 102. In addition, in an undeflected state, the tubular shaft 102 may define mutually perpendicular planes (e.g. cartesian planes) extending through and intersecting along the longitudinal axis of the tubular shaft 102. In one embodiment, as shown in FIG. 1, the mutually perpendicular planes are represented by the axes x-y and y-z, with the tubular shaft 102 defining the axis y.

In certain instances, the tubular shaft 102 extends from a distal portion of a handle 112. An electrical cable or other suitable connector 114 extending from a proximal end of the handle 112 may be coupled to a source of energy or other equipment (not shown in FIG. 1) for transmitting one or more ablation signals. FIG. 1 generally illustrates one or more ablation electrodes 116 disposed at the distal end 110 of the distal portion 106.

A steering actuator 118, such as a rotatable knob or plunger that may be arranged at a distal end of the handle 112, may be manipulated by a physician to deflect or position the steerable distal portion 106 of the tubular shaft 102.

As shown in FIGS. 2-3, the electrophysiology catheter 100 is of the deflectable or steerable type, such that during use, the deflection region 108 can be deflected or curved by a user to facilitate locating the distal end 110 and the ablation electrodes 116 at a desired target location within the heart. In embodiments, deflection of the deflection region 108 can be accomplished by manipulation of the steering actuator 118, which is operatively connected to steering elements (e.g., wires, tendons, ribbons, and the like) extending within and attached (directly or indirectly) to the shaft 102 at a location within the distal portion 106. The particular mode and structure for causing the deflection the deflection region 108 is not critical to the present disclosure, and so any technique, whether now known or later developed, can be employed within the scope of the present disclosure.

In use, deflecting or curving the distal portion 106 may impart a torsional force that could torque or twist the distal portion 106 away from or out of the target location. The deflection region 108 may torque out of plane from the plane in which the distal portion 106 was arranged prior to deflection due to the tension on the curvature of the electrophysiology catheter 100 within vasculature. As will be discussed in more detail below, the catheter 100 is configured such that the planarity of the deflection region 108 is maintained substantially along the y-z plane.

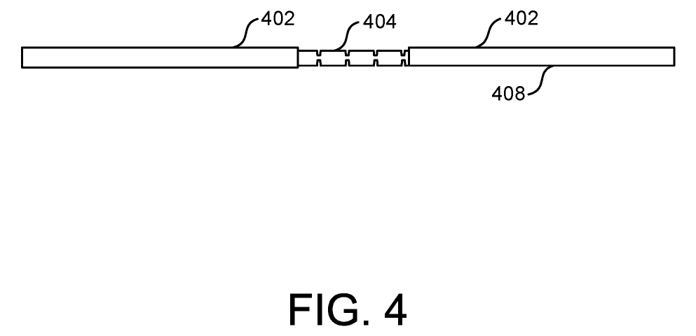

FIG. 4 is side view of a portion of the shaft 102 including the deflection region 108, consistent with various aspects of the present disclosure. As shown, shaft 102 includes an outer tubular jacket 402 (shown partially cut away in FIG. 4), and an articulation member 404 disposed within the outer tubular jacket 402.

In certain instances, the outer tubular jacket 402 is of a braided construction having a polymer material reinforced with a metal or polymeric braid formed of a plurality of interwoven wires that are woven, knitted, entwined or otherwise interlaced together. The skilled artisan will recognize that the use of braided jackets in catheter construction is well known, and the particular details of the braid/jacket construction are not of critical importance to the present disclosure. In embodiments, the aforementioned braid can be omitted, or alternatively, other constructions, e.g., reinforcing coils, can be employed to enhance the structural and torsional strength of the jacket 402.

As will be explained in greater detail below, and with reference to FIGS. 2-3, the articulation member 404 is configured to exhibit a relatively high degree of flexibility in the y-z plane, while at the same time being relatively inflexible in the x-y plane. As such, the articulation member 404 facilitates predictable, highly planar deflection of the deflection region 108 by resisting torsional forces on the shaft 102 that would otherwise tend to cause the deflection region 108 to deflect or bend in the x-y plane (or some other plane oriented transversely to the y-z plane).

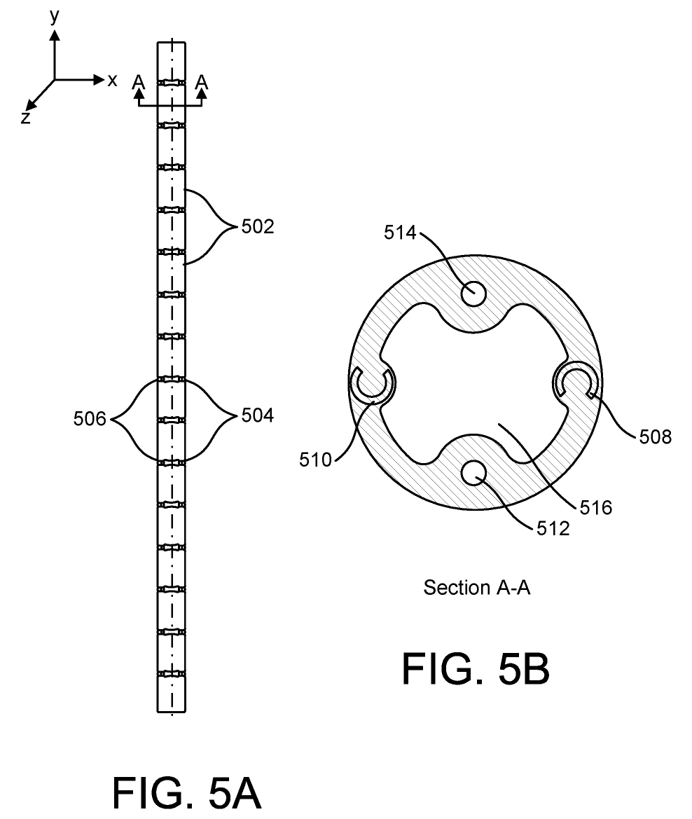

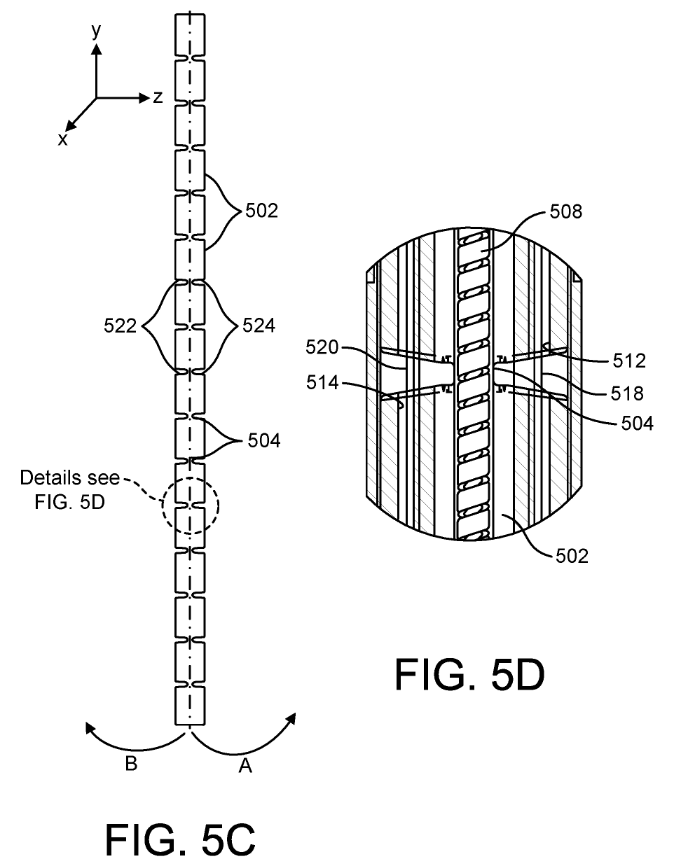

FIG. 5A is a top-view illustration of an articulation member 404 of the electrophysiology catheter 100, consistent with various aspects of the present disclosure. As shown, the articulation member 404 includes a plurality of longitudinally arranged tubular segments 502, a plurality of connecting segments 504, and a plurality of connecting segments 506. The adjacent tubular segments 502 are joined by respective ones of the connecting segments 504, 506. As shown, the connecting segments 504, 506 are disposed in the x-y plane when the articulation member 404 is in an undeflected state, represented by the cartesian coordinate system shown along with FIG. 5A.

FIG. 5B is a cross-sectional illustration of the tubular segments 502 of the electrophysiology catheter 100 taken along the line A-A in FIG. 5A, consistent with various aspects of the present disclosure. As shown, the articulation member 404 includes a pair of reinforcing members 508, 510 extending therethrough. As further shown, the reinforcing members 508, 510 are embedded within the connecting segments 504, 506, respectively, to strengthen the connections between adjacent tubular segments 502, as will be discussed in further detail below.

As shown, the tubular segments 502 may include a pair of steering wire lumens 512, 514 arranged in a plane defined by axes y and z, perpendicular to the x-y plane in which the connecting segments 504, 506 lie. As one skilled in the art would appreciate, the steering wire lumens 512, 514 are configured to receive steering wires or members that are operatively connected to the steering actuator 118 (FIG. 1) to facilitate the deflection of at least the deflection region 108 of the tubular shaft 102 as known in the conventional manner. In some embodiments, the segments 502 may include only one steering wire lumen.

As shown, the steering wire lumens 512, 514 (and consequently, the steering wires received within each, respectively) are located at diametrically opposite positions from one another about the circumference of the articulation member 404. As such, the steering wire lumens 512, 514 are circumferentially offset from one another by about 180 degrees and are each circumferentially offset from the reinforcing members 508, 510 by about 90 degrees. In this configuration, the steering wire lumens 512, 514 lie in the y-z plane.

FIG. 5C is a side-view illustration of the articulation member 404 of the electrophysiology catheter 100, consistent with various aspects of the present disclosure. As shown, the tubular segments 502 are connected through the connecting segments 504, 506 disposed in the plane defined by axes x and y, represented by the cartesian coordinate system shown along with FIG. 5C. The articulation member 404 shown in FIG. 5C is 90 degrees offset from the articulation member 404 shown in FIG. 5A, which is also demonstrated by the rotation of the x and z axes in the two figures.

The connecting segments 504, 506 function as living hinges integrally formed with the tubular segments 502. In response to a deflection force, the articulation member 404 may bend in a direction A along the y-z plane. In certain embodiments, the articulation member 404 may bend in another direction B, opposite to the direction A along the y-z plane.

Regardless of which direction (e.g., A or B) the articulation member 404 bends, the living hinges create a plurality of slits 522 and a plurality of slits 524 in between each adjacent tubular segments 502 so as to articulate the bending to be substantially within the y-z plane. In some embodiments, the plurality of slits 522, 524 between different adjacent tubular segments 502 can be different (e.g., with respect to spacing, lengths, and/or shapes) to fine-tune articulation of the distal portion 106 of the tubular shaft 102. In some embodiments, the slits 522, 524 may be concave and substantially “V-shaped,” or substantially “U-shaped.”

The living hinge design enables the articulation member 404 to exhibit a relatively high degree of flexibility in the y-z plane, while at the same time being relatively inflexible in the x-y plane (the plane passing through the living hinges). As such, the plurality of slits 522, 524 facilitate the articulation member 404 to have predictable, highly planar deflection of the deflection region 108 by resisting torsional forces on the shaft 102 that would otherwise tend to cause the deflection region 108 to deflect or bend in the x-y plane (or some other plane oriented transversely to the y-z plane).

FIG. 5D is a detailed illustration of a portion of a side-view illustration of the articulation member 404 of the electrophysiology catheter 100, consistent with various aspects of the present disclosure. As shown, the reinforcing member 508 is embedded within the connecting segments 504, and within the tubular segments 502. In addition, the steering wires 518, 520 are located in the steering wire lumens 512, 514.

In embodiments, the materials used for the living hinges can be relatively soft to minimize the potential for stress-induced failures of the connecting segments 504, 506. Softer material deforms easily during articulating, thus limiting ability for fine movements and decreasing overall durability. Therefore, the reinforcing members 508, 510 are made of stiffer materials than the connecting segments 504, 506. In some embodiments, the reinforcing members 508, 510 each includes a helically wound flat ribbon wire. In certain instances, the reinforcing members 508, 510 may include coils, stranded wire cables, stiffer polymer materials, and other metal or polymer components. In other instances, the materials used for the reinforcing members 508, 510 can be altered according to the specific needs of the application.

In embodiments, the tubular segments 502 may include a center lumen 516 arranged centrally within the tubular segments 502 along the tubular shaft 102. In certain instances, the center lumen 516 may include components such as one or more wires for ablation electrodes arranged along the tubular shaft 102, navigational components, a temperature sensor (e.g., thermocouple), a force sensor, radio-frequency circuitry and/or wires, and a cooling lumen. The center lumen 516 may be a working channel through which one or more devices may be passed.

As shown, the center lumen 516 may be of an hourglass shape. In some embodiments, the center lumen 516 may be substantially of a round or circular shape. The pair of steering wire lumens 512, 514 may be arranged on opposite sides of the center lumen 516.

In certain instances, the steering wire lumens 512, 514 may extend along the y-z plane and may be aligned with the x-y plane. The steering wire lumens 512, 514 are configured to receive steering elements (e.g., wires, tendons, ribbons made of stainless steel, titanium, MP35N, a suitable alloy, and other suitable materials) to apply deflection forces to deflect or curve at least the deflection region 108 of the tubular shaft 102.

The deflection force may twist or torque the deflection region 108 out of approximately the y-z plane due to tension on the steering wire lumens 512, 514 and/or the curvature of the electrophysiology catheter 100 within vasculature. The connecting segments 504, 506 are configured to maintain planarity of the deflection region 108 in response to curvature of the deflection region 108. The connecting segments 504, 506 may stabilize the deflection region 108. For example, the connecting segments 504, 506 may create a bias at the deflection region 108 to substantially align with the deflection. In certain instances, the connecting segments 504, 506 may resist bending out of the y-z plane. The connecting segments 504, 506 are configured to maintain the deflection region 108 approximately within the y-z plane (e.g., within approximately +/- 10% of the plane) while the deflection force curve or deflect at least the distal portion 106 of the tubular shaft 102. In certain instances, the connecting segments 504, 506 are configured to maintain the deflection region 108 substantially aligned (e.g., within approximately +/- 10%) with the y-z plane in response to the deflection force.

The connecting segments 504, 506 may maintain planarity of the deflection region 108 and lessen reliance of bending planarity on the relatively less stiff (e.g., polymer materials) of the tubular segments 502 of the tubular shaft 102. The connecting segments 504, 506 may maintain a curved shape of the deflection region 108 during deflection and maintain repeatability of achieving the curved shape of the deflection region 108 during deflection.

In embodiments, the plurality of tubular segments 502 may be substantially of the same length. In other embodiments, the length of the tubular segments 502 may be varied to customize or fine tune the deflection shape of at least the deflection region 108 of the tubular shaft 102.

In some instances, the reinforcing members 508, 510 are embedded within the tubular segments 502 through direct overmolding to form a bond between the tubular segments 502 and the connecting members 504, 506. The bond between the tubular segments 502 and the connecting members 504, 506 may be further strengthened through adhesives, surface treatments, or other applications that may enhance the bond.

Direct overmolding allows for high-strength joints between the adjacent tubular segments 502 that still maintain desired flexibility. In some embodiments, the reinforcing members 508, 510 have higher surface area due to the helically wound flat ribbon shape to allow for stronger mechanical bond or lamination into the molded connecting segments 504, 506.

Thus, the plurality of tubular segments 502 may be made of stiffer materials to increase durability and articulation feedback during use. For instance, the tubular segments 502 may be made of polycarbonate (PC), acrylonitrile butadiene styrene (ABS), PC/ABS blends, polyetheretherketone (PEEK), acetal, polyetherimide, or liquid crystal polymer (LCP).

The direct overmolding process greatly decreases processing time and operator interaction compared to stringing adjacent tubular segments 502 over the connecting segments 504, 506 separately. This process allows lower inherent variation by creating the entire articulation member 404 in one process. The mold can be sized to allow the reinforcing members 508, 510 to pass along length of part while preventing significant flow of material through the connecting segments 504, 506. The constraints at the connecting segments 504, 506 allow them to be molded with or without tension. In addition, tab or oval-shaped gates are used at parting line to allow flow through and/or around the reinforcing members 508, 510, thus achieving proper containment. The reinforcing members 508, 510 prevent compression during articulation, leading to greater consistency after higher cycle counts.

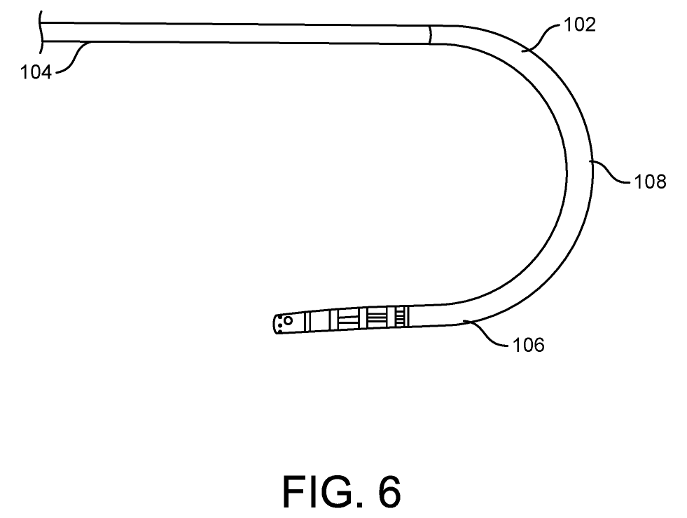

FIG. 6 is another illustration of an example electrophysiology catheter 100, consistent with various aspects of the present disclosure. As discussed in detail above, the electrophysiology catheter 100 includes a tubular shaft 102 having a deflection region 108. The tubular shaft 102 includes an outer tubular jacket 402. In some embodiments, the outer tubular jacket 402 may include multiple layers (e.g., an outer layer made of TPE, TPU such as PEBA, or other material with a durometer less than 40D, and a braided middle layer of high coverage, high pick-per-inch (PPI), multi-wire braid).

The articulation member 404 (shown in FIG. 5A-D) is disposed within the deflection region 108. The articulation member 404 facilitate maintaining planarity of the curvature of the deflection region 108. For example, the articulation member 404 is configured to create a force perpendicular to the deflection (e.g., caused by the deflection force) creating a bend between the pair of reinforcing members 508, 510.

The articulation member 404 being arranged within the deflection region 108 of the tubular shaft 102 operates to stabilize at least the distal portion 106 of the tubular shaft 102. In other instances, the articulation member 404 may extend along a length of the tubular shaft 102 or extend into an intermediate section between the proximal portion 104 and the distal portion 106.

The articulation member 404 embedded within the deflection region 108 of the tubular shaft 102 may be caused to assume a curved shape when the tubular shaft 102 is arranged within a patient’s vasculature. The ability to selectively curve the deflection region 108 can facilitate accurate and effective delivery therapy (e.g., ablation) or map the surface of the heart tissue (e.g., identify the locations of heart tissue that are a source of the arrhythmias).

In embodiments, the braided structure can operate to reinforce the connecting segments 504, 506 during torsional or tensile loading. The combination of high PPI braid for a middle layer and low durometer material for an outer layer may increase elasticity of the outer tubular jacket 402. The high elasticity of the outer tubular jacket 402 prevents undue stress during articulation by reducing compression while stretching the outer tubular jacket 402. However, the use of braided jackets in catheter construction is well known in the art, and the particular details of the braid/jacket construction are not of critical importance to the present disclosure. In embodiments, the aforementioned braid can be omitted, or alternatively, other constructions, e.g., reinforcing coils, can be employed to enhance the structural and torsional strength of the jacket 402.

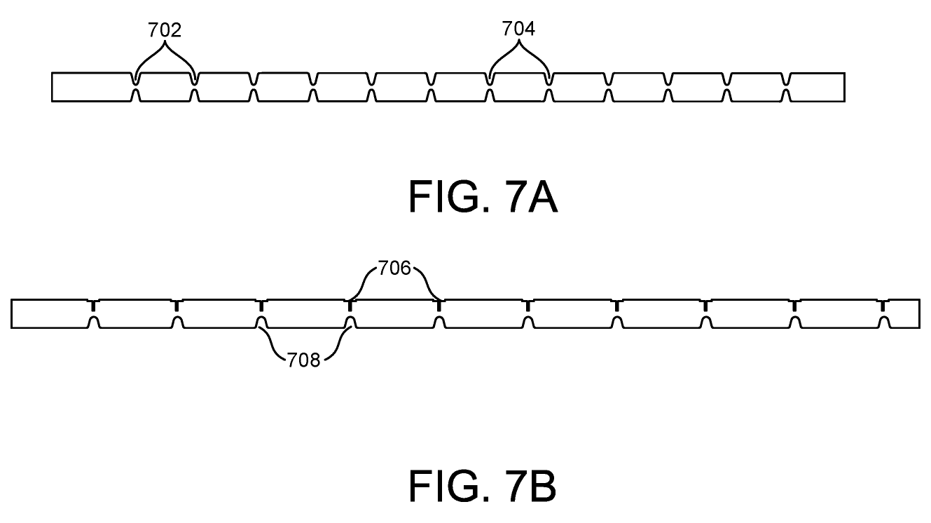

FIG. 7A is an embodiment of a side-view illustration of a portion of the articulation member 404 according to another embodiment of the disclosure. As shown, the plurality of slits 702 are of a different shape than the plurality of slits 704 so as to allow different bend radii in the regions of the articulation member 404 where each of the plurality of slits are located. For example, in the illustrated embodiment, the slits 702 are narrower than the slits 704. As a result, when a deflection force is applied so as to cause the articulation member 404 to bend, the bend radius of the portion of the articulation member 404 containing the slits 702 will be larger than the bend radius of the portion containing the slits 704. Accordingly, selectively providing slits 702, 704 of different widths along different portions of the articulation member 404 can allow the articulation member 404 achieve multi-radius curvatures.

FIG. 7B is an embodiment of a side-view illustration of a portion of an alternative embodiment of the articulation member 404 that facilitates assumption of unidirectional or asymmetric curves in the articulation member 404. As shown, the plurality of slits 706 are substantially closed gaps, e.g., the opposing tubular segment faces spanned by the slits 706 are in contact, or spaced very closely, when the articulation member 404 is in its undeflected state. As further shown, the slits 708 are relatively wide. In this embodiment, the articulation member 404 is substantially inflexible in the direction of bending toward the slits 706, while still allowing the articulation member 404 to bend in the direction of the slits 708. The articulation member 404 as shown in this figure would be relatively straight and undeflected in the direction towards the side where the narrow slits 706 are, so as to achieve substantially unidirectional bending. In other embodiments, the slits 706 and 708 can be selectively located along the articulation member 404 so as to allow the articulation member 404 to assume asymmetric bending profiles, e.g., wherein the deflection point in the bending direction toward the slits 706 is different than the deflection point in the bending direction toward the slits 708. The skilled artisan will readily appreciate that still different configurations of the articulation member 404 and the slits 706, 708 can be employed to further tune the bending profile as desired.

In other embodiments, slits of different cut width, depth, shape, and locations on either side of the articulation member 404 can be changed to fine tune the curve characteristics, thus creating asymmetry in the bending or articulation.

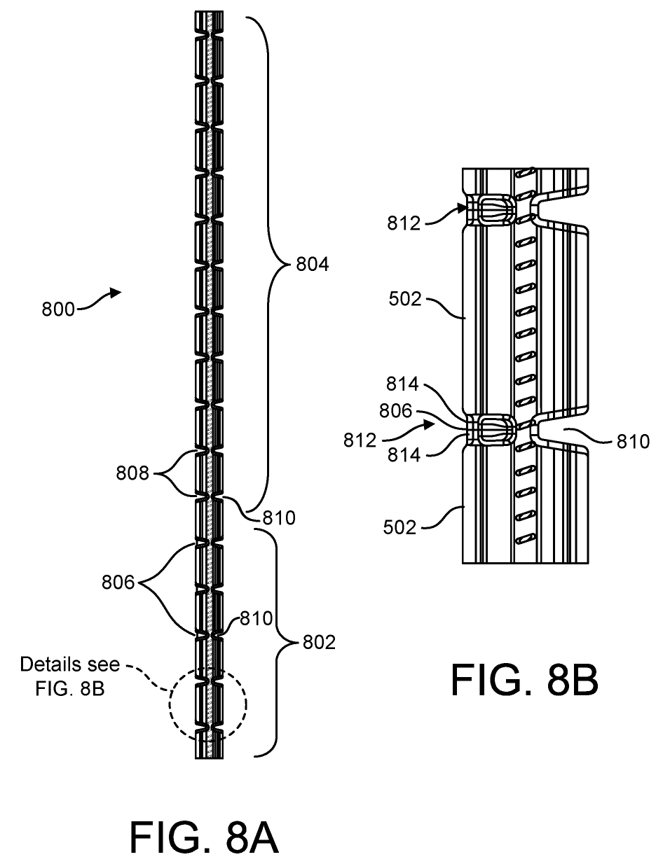

FIG. 8A is a side-view illustration of a portion of an alternative embodiment of the articulation member 800. As shown, in the embodiment of FIG. 8A, the articulation member 800 includes a proximal articulation region 802 and a distal articulation region 804. Similar to other embodiments of the disclosure, the articulation member 800 includes a plurality of slits 806 extending between adjacent tubular segments along one circumferential side of the articulation member 800 in the proximal articulation region 802, and slits 808 extending between adjacent tubular segments along the same circumferential side of the articulation member 800 in the distal articulation region 804. As further shown, a plurality of slits 810 extend between adjacent tubular segments along the circumferential side opposite the slits 806, 808 along the length of the articulation member 800. Respective slits 806, 810 and 808, 810 at the same longitudinal location form slit pairs that are separated by the connecting segments between the adjacent tubular segments 502.

In embodiments, as will be explained in greater detail below, the proximal articulation region 802 can articulate substantially unidirectionally while the distal articulation region 804 can articulate bidirectionally. This is because the plurality of slits 806 that are located solely in the proximal region 802 are substantially closed gaps. Such a configuration is in contrast to slits 808 and 810 that are relatively wide (e.g., at least ten times wider in the direction of the longitudinal axis). In this embodiment, the proximal region 802 of articulation member 800 is substantially inflexible in the direction of bending toward the slits 806, but the distal region 804 is allowed to bend in the direction of slits 808. In addition, the whole length of the articulation member 800 is allowed to bend in the direction of the slits 810. Effectively, such an arrangement shortens the length of the articulation member 800 in one direction. In embodiments, the length of the articulation member 800 that can bend to the left (as shown in FIG. 8A) is shorter than the length of the articulation member 800 that is allowed to bend to the right (as shown in FIG. 8A). In some embodiments, the proximal region 802 is about one third of the length of the distal region 804.

FIG. 8B is a detailed illustration of a portion of a side-view illustration of the alternative embodiment of the articulation member 800. As shown, the articulation member 800 includes limiting features 812 (although only two are visible in FIG. 8B) that are solely located in the proximal region 802. In the illustrated embodiment, each limiting feature 812 includes a pair of extensions 814 that are located on two adjacent tubular segments 502. Each extension 814 is in contact with, or spaced very closely to, its corresponding opposing extension 814 when the articulation member 800 is in its undeflected state. Thus, the slits 806 are very narrow, or essentially of zero slit width, compared to the slits 808, 810. In this embodiment, the proximal region 802 of the articulation member 800 is substantially inflexible in the direction of articulating toward the slits 806, while still allowing the proximal region 802 of the articulation member 800 to articulate in the direction of the slits 810.

In embodiments, the extensions 814 can be formed as a web of molding material that is subsequently mechanically cut (e.g., via a laser cutting process) to form the corresponding slit 806. In the illustrated embodiment, the extensions 814 are recessed relative to the outer surfaces of the tubular segments 502 as a result of the particular mold design, which reduces the thickness of the web that must be cut to form the slits 806. However, the skilled artisan will recognize that the particular geometry of the extensions 814 shown in FIG. 8B are exemplary only.

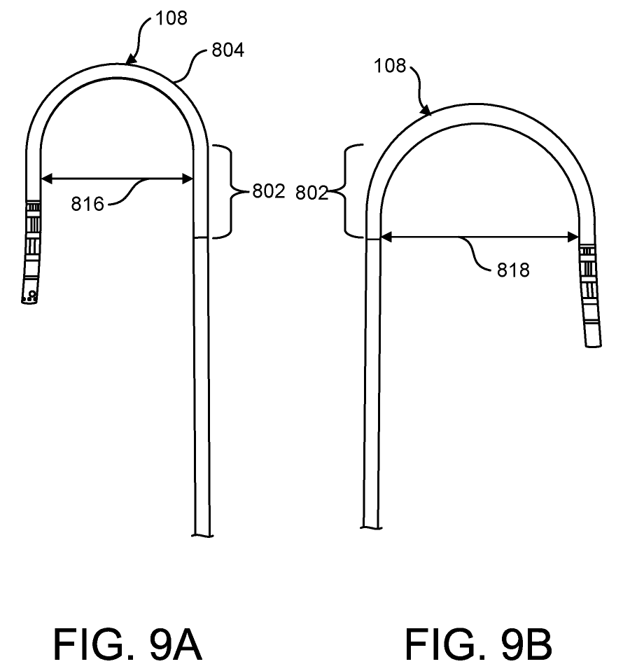

FIGS. 9A and 9B illustrate an embodiment of the tubular shaft 102 that incorporates the articulation member 800. As shown in FIG. 9A, when a deflection force is applied by steering wires extending through the circumferential side of the articulation member having the limiting features 812 (i.e., the extensions 814 forming closed slits 806 in the undeflected state), the deflection region 108 assumes a first curved shape, wherein it begins to curve at a transition between the proximal articulation region 802 and the distal articulation region 804 of the articulation member 800. In contrast, FIG. 9B illustrates the distal portion of the catheter shaft when a deflection force is applied by steering wires extending through the circumferential side of the articulation member having the slits 810 (i.e., slits without the limiting features 812), such that the deflection region 108 assumes a second shape, wherein it begins to curve proximate the proximal end of the proximal articulation region 802 such that substantially the entire length of the deflection region 108 assumes the second curved shape.

The result is that the curve diameter 816 of the distal portion shown in FIG. 9A is smaller than the curve diameter 818 of the distal portion shown in FIG. 9B. In embodiments, the curve diameter 816 can range from about 8 mm to about 55 mm, whereas the curve diameter 818 can range from about 8 mm to about 80 mm. Thereby, the same catheter 100 can have different steering curvatures depending on the which direction the physician orients the catheter 100. In some embodiments, the curve diameter 816 is about thirty percent tighter than the curve diameter 818. The difference in curve diameters 816, 818 can aid the physician in navigating the various patient anatomy and may prevent the need to use multiple different catheters for a single procedure.

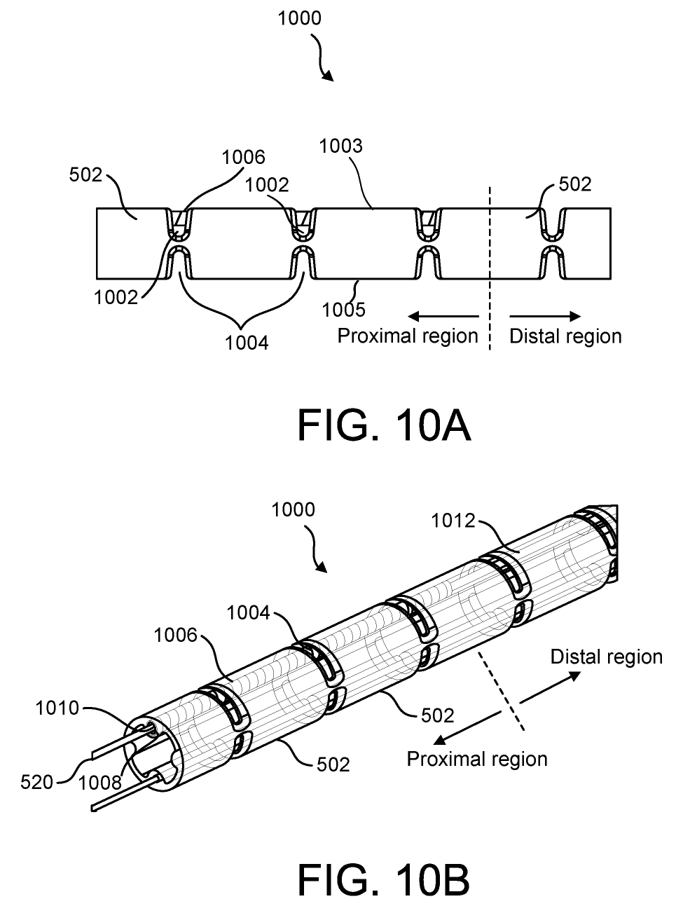

FIG. 10A is a side-view illustration of a portion of an alternative embodiment of the articulation member 1000 in an undeflected state. As shown, the articulation member 1000 includes a proximal articulation region and a distal articulation region. Similar to other embodiments of the disclosure, the articulation member 1000 includes a plurality of slits 1002 extending between adjacent tubular segments 502 along one circumferential side 1003 of the articulation member 1000 along the length of the articulation member 1000, and a plurality of slits 1004 extending between adjacent tubular segments 502 along the circumferential side 1005 opposite the slits 1002 along the length of the articulation member 1000. Respective slits 1002, 1004 at the same longitudinal location form slit pairs that are separated by the connecting segments between the adjacent tubular segments 502. In embodiments, the slits 1002, 1004 may have substantially equal slit widths, although in other embodiments the slit widths of one or both of the slits 1002, 1004 may vary along the length of the articulation member 1000.

As further shown in FIG. 10A, the articulation member 1000 includes a single coil member 1006 extending through the tubular segments 502 in only the proximal region along the circumferential side 1003. In the illustrated embodiment, the coil member 1006 is in the form of a close-pitched coil member (e.g., a Bowden coil or a coil spring). The coil member 1006 can be formed from a flat ribbon wire that is helically wound to have a close pitch when in an undeflected state, where each coil turn is in contact with, or is spaced very closely to, its adjacent coil turns so as to be essentially non-compressible under compressive loads while being extendable under tensile loads. Thus, articulation of the articulation member 1000 towards the coil member 1012 will be prevented. In contrast, when a deflection force is applied away from the coil member 1006, the coil member 1006 will elastically deform. During which, the coils with move apart from each other, essentially increasing the length of the coil member 1006. This allows for articulation of the articulation member 404 towards the circumferential side that is opposite from the coil member 1006. Thereby, this embodiment of the articulation member 1000 can have the same or similar articulation behavior as shown in FIGS. 9A and 9B when incorporated into a catheter shaft.

FIG. 10B is a perspective view illustration of the alternative embodiment of the articulation member 1000. As shown, the coil member 1006 is positioned in bore 1008 which extends through each of the tubular members 502 in the proximal region. The coil member 1006 includes a steering wire lumen 1010, in which the steering wire 520 is located. At the end of the steering wire lumen 1010, the steering wire lumen 1012 begins. The steering wire lumen 1012 allows the steering wire 520 to extend through the tubular members 502 in the distal region of the articulation member 1000.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claimed is:

1. A medical device comprising:
a handle; and
a tubular shaft having a proximal portion extending from the handle, and a distal portion having a distal end and a deflection region, the shaft defining a longitudinal axis and including:
an outer tubular jacket; and
an articulation member disposed within the jacket in the deflection region, the articulation member having a proximal articulation region and a distal articulation region and comprising a plurality of longitudinally-arranged tubular segments, a plurality of first connecting segments, and a plurality of second connecting segments, wherein adjacent tubular segments are joined by respective ones of the first and second connecting segments, and wherein all of the first and second connecting segments are disposed in a first plane extending through the longitudinal axis, and wherein a plurality of diametrically opposed slit pairs are disposed longitudinally along the articulation member, each slit pair separating adjacent tubular segments between respective ones of the first and second connecting segments and including a first slit and a second slit, wherein the slit pairs are centered on a second plane that is orthogonal to the first plane; and
wherein the proximal articulation region is configured to articulate in a first direction within the second plane, and to resist articulation in a second direction within the second plane, the second direction being opposite the first direction, and
wherein the distal articulation region is configured to articulate in both the first direction and the second direction.

2. The medical device of claim 1, further comprising:
first and second steering wire lumens extending through the tubular shaft to a location distally beyond the articulation member and within the second plane; and
first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

3. The medical device of claim 2, wherein the articulation member includes a first reinforcing member and a second reinforcing member extending through the plurality of first and second connecting segments, respectively, wherein the first and second reinforcing members are embedded, respectively, within the first and second connecting segments and within the tubular segments.

4. The medical device of claim 3, wherein:
the deflection region is configured to assume a first curved shape when the first deflection force is applied to the distal portion of the shaft;
the deflection region is configured to assume a second curved shape when the second deflection force is applied to the distal portion of the shaft, wherein the second curved shape is different than the first curved shape; and
the first curved shape has a first curve diameter, and the second curved shape has a second curve diameter, wherein a first radius of curvature is greater than a second radius of curvature.

5. The medical device of claim 3, wherein the articulation member is configured such that the deflection region begins to articulate in the first direction proximate a proximal end of the deflection region when the first deflection force is applied to the distal portion of the shaft; and the deflection region begins to articulate in the second direction at a transition between the proximal articulation region and the distal articulation region when the second deflection force is applied to the distal portion of the shaft.

6. The medical device of claim 3, wherein the articulation member is configured such that the deflection region begins to articulate in the first direction at a first location along the shaft when the first deflection force is applied to the distal portion of the shaft; and the deflection region begins to articulate in the second direction at a second location along the shaft when the second deflection force is applied to the distal portion of the shaft, wherein the first location is proximal to the second location.

7. The medical device of claim 1, wherein each of the slit pairs includes a first slit having a first slit width located at a first circumferential side of the articulation member, and a second slit having a second slit width located on a second circumferential side of the articulation member.

8. The medical device of claim 1, wherein the slit pairs in the proximal articulation region are configured to permit articulation of the proximal articulation region in the first direction within the second plane, and to resist articulation of the proximal articulation region in the second direction within the second plane.

9. The medical device of claim 8, wherein:
when the deflection region is in an undeflected state, the second slit width is substantially zero such that adjacent tubular segments contact each other along the first circumferential side, and the first slit width larger than the second slit width;
each adjacent tubular segment in the proximal deflection region includes a limiting feature that is configured to resist articulation of the proximal articulation region in the second direction; and
the limiting feature includes a pair of opposed extensions positioned respective adjacent tubular segments, wherein each of the extensions is configured to contact the other extension to resist articulation of the proximal articulation region in the second direction, and to move away from the other extension to allow deflection of the proximal articulation region in the first direction.

10. The medical device of claim 8, wherein the articulation member includes a coil member extending through the tubular segments of the proximal articulation region within the second plane, the coil member configured to allow articulation of the proximal articulation region in the first direction, and to resist articulation of the proximal articulation region in the second direction.

11. A medical device comprising:
a handle; and
a tubular shaft having a proximal portion extending from the handle, and a distal portion having a distal end and a deflection region, the shaft defining a longitudinal axis and including an articulation member disposed in the deflection region, the articulation member having a proximal articulation region and a distal articulation region and comprising a plurality of longitudinally-arranged tubular segments and a plurality connecting segments, wherein adjacent tubular segments are joined by respective ones of the connecting segments, and wherein all of the connecting segments are disposed in a first plane extending through the longitudinal axis, and wherein a plurality of diametrically opposed slit pairs are disposed longitudinally along the articulation member, each slit pair separating adjacent tubular segments between respective ones of the connecting segments, wherein the slit pairs are centered on a second plane that is orthogonal to the first plane, wherein:
the proximal articulation region is configured to articulate in a first direction within the second plane, and to resist articulation in a second direction within the second plane, the second direction being opposite the first direction; and
the distal articulation region is configured to articulate in both the first direction and the second direction; and
first and second steering wire lumens extending through the tubular shaft to a location distally beyond the articulation member and within the second plane; and
first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

12. The medical device of claim 11, wherein:
the deflection region is configured to assume a first curved shape when the first deflection force is applied to the distal portion of the shaft; and
the deflection region is configured to assume a second curved shape when the second deflection force is applied to the distal portion of the shaft, wherein the second curved shape is different than the first curved shape.

13. The medical device of claims claim 11, wherein each of the slit pairs includes a first slit having a first slit width located at a first circumferential side of the articulation member, and a second slit having a second slit width located on a second circumferential side of the articulation member.

14. The medical device of claim 13, wherein the slit pairs in the proximal articulation region are configured to permit articulation of the proximal articulation region in the first direction within the second plane, and to resist articulation of the proximal articulation region in the second direction within the second plane.

15. The medical device of claim 13, wherein the articulation member includes a coil member extending through the tubular segments of the proximal articulation region within the second plane, the coil member configured to allow articulation of the proximal articulation region in the first direction, and to resist articulation of the proximal articulation region in the second direction.

16. A medical device comprising:
an outer tubular jacket; and
an articulation member disposed within the jacket, the articulation member having a proximal articulation region and a distal articulation region and comprising a plurality of longitudinally-arranged tubular segments, a plurality of first connecting segments, and a plurality of second connecting segments, wherein adjacent tubular segments are joined by respective ones of the first and second connecting segments, and wherein all of the first and second connecting segments are disposed in a first plane extending through the longitudinal axis, and wherein a plurality of diametrically opposed slit pairs are disposed longitudinally along the articulation member, each slit pair separating adjacent tubular segments between respective ones of the first and second connecting segments and including a first slit and a second slit, wherein the slit pairs are centered on a second plane that is orthogonal to the first plane; and
wherein the proximal articulation region is configured to articulate in a first direction within the second plane, and to resist articulation in a second direction within the second plane, the second direction being opposite the first direction,
wherein the distal articulation region is configured to articulate in both the first direction and the second direction, and
wherein each of the slit pairs includes a first slit having a first slit width located at a first circumferential side of the articulation member, and a second slit having a second slit width located on a second circumferential side of the articulation member.

17. The medical device of claim 16, wherein the slit pairs in the proximal articulation region are configured to permit articulation of the proximal articulation region in the first direction within the second plane, and to resist articulation of the proximal articulation region in the second direction within the second plane.

18. The medical device of claim 17, wherein when the articulation member is in an undeflected state, the second slit width is substantially zero such that adjacent tubular segments contact each other along the first circumferential side, and the first slit width larger than the second slit width.

19. The medical device of claim 16, wherein the articulation member includes a coil member extending through the tubular segments of the proximal articulation region within the second plane, the coil member configured to allow articulation of the proximal articulation region in the first direction, and to resist articulation of the proximal articulation region in the second direction.

20. The medical device of claim 16, further comprising:
first and second steering wire lumens extending through the tubular shaft to a location distally beyond the articulation member and within the second plane; and
first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.
